(19) United States
(12) Patent Application Publication
(10) Pub. No.: US 2022/0296618 A1
(43) Pub. Date: Sep. 22, 2022

(54) ANTI-INFLAMMATORY COMPOUNDS FOR USE IN THE TREATMENT OF DERMAL DISORDERS

(71) Applicant: COUNCIL OF SCIENTIFIC & INDUSTRIAL RESEARCH, New Delhi (IN)

(72) Inventors: Srigiridhar KOTAMRAJU, Hyderabad, Telangana (IN); Surendar Reddy BATHULA, Hyderabad, Telangana (IN); Ramakrishna SISTLA, Hyderabad, Telangana (IN); Anthony ADDLAGATTA, Hyderabad, Telangana (IN); Rajamannar THENNATI, Vadodara, Gujarat (IN); Altab SHAIKH, Hyderabad, Telangana (IN); Aruna JANGUM, Hyderabad, Telangana (IN); Rajendra SANGARAJU, Hyderabad, Telangana (IN); Praveen Kumar NEELI, Hyderabad, Telangana (IN); Kuncha MADHUSUDANA, Hyderabad, Telangana (IN); Singuru GAJALAKSHMI, Hyderabad, Telangana (IN)

(21) Appl. No.: 17/755,556

(22) PCT Filed: Nov. 23, 2020
(86) PCT No.: PCT/IB2020/061043

§ 371 Date: May 2, 2022

(30) Foreign Application Priority Data
Nov. 22, 2019 (IN) 201911047906

(51) Int. Cl. A61K 31/665; A61K 9/00; A61P 17/02

(52) U.S. Cl. CPC A61K 31/665; A61K 9/0014; A61P 17/02

(57) ABSTRACT

The present invention relates to the use of a compound of Formula (I) for the treatment of a wound, impaired healing of a wound, hair loss on and around a wound, scars and/or wrinkles on and around a wound, psoriasis, and any combination thereof. The present inventions also relates to methods of using such compounds, and to compositions and kits containing the compound. (I) X⁻ is any halogen.

This application claims the benefit of Indian Patent Application No. 201911047906, filed Nov. 22, 2019, which is hereby incorporated by reference.

### FIELD OF THE INVENTION

The present invention relates to a compound of Formula I (shown below) for use in the treatment of one or more conditions selected from the group consisting of a wound, impaired healing of a wound, hair loss on and around a wound, scars and/or wrinkles on and around a wound, and psoriasis, as well as topical compositions containing a compound of Formula I and a pharmaceutically acceptable excipient.

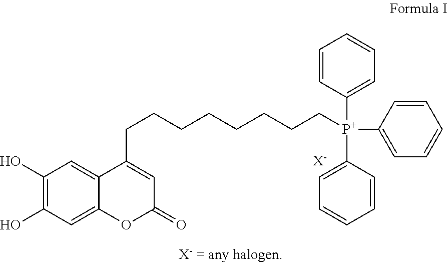

### BACKGROUND OF THE INVENTION

Oxidative stress is one of the prime events that hamper the tissue repair and regeneration process. Scavenging of over accumulated free radicals elicits a faster healing process, possibly by promoting prerequisite angiogenesis and new blood vessel formation at the wound site.

Coumarins consist of a group of phenolic compounds widely distributed in natural plants and they possess a wide range of pharmacological activities. See, e.g., Egan et al., Drug Metab. Rev., 22: 503-529, 1990. Of these, esculetin (6,7-dihydroxycoumarin) (Compound 2) has been reported to lower serum levels of the hepatic enzyme markers ALT (alanine aminotransferase) and AST (aspartate aminotransferase) when administered intraperitoneally prior to treatment with t-butyl hydroperoxide. See, e.g., Lin et al., Arch. Toxicol., 74, 467-72, 2000.

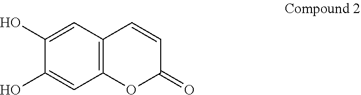 [SMILES: O=c1ccc2cc(O)c(O)cc2o1]

However, as coumarins typically have poor bioavailability in vivo and do not significantly accumulate within mitochondria, their effectiveness remains limited. Due to this, coumarins typically have to be employed in higher concentrations to scavenge mitochondrial reactive oxygen species.

U.S. Pat. No. 9,580,452 discloses a triphenylphosphonium cation (TPP+) coupled esculetin of formula I (shown below) having an anti-atherosclerotic effect.

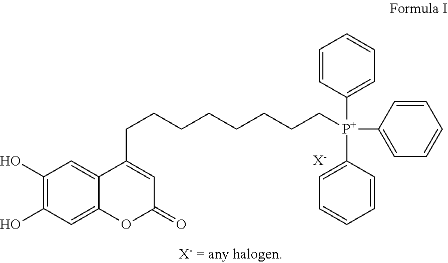

There is a need for improved treatments of wound related disorders and psoriasis.

### SUMMARY OF THE INVENTION

The present inventors have surprisingly found that a compound of formula I may be used for the treatment of various wound related conditions and psoriasis. In one preferred embodiment, the compound of formula I is administered topically.

One embodiment is a compound of formula I for use in treating one or more of a wound, impaired healing of a wound, hair loss on and around a wound, scars and/or wrinkles on and around a wound and psoriasis.

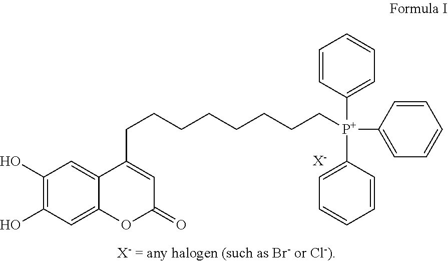

In one preferred embodiment, X⁻ is Br⁻ or Cl⁻. In a more preferred embodiment, X⁻ is Br⁻.

Another embodiment is a method of treating a wound, impaired healing of a wound, hair loss on and around a wound, scars and/or wrinkles on and around a wound, psoriasis, and any combination thereof in a subject in need thereof. The method comprises administering (preferably topically) to the subject a therapeutically effective amount of a compound of formula I:

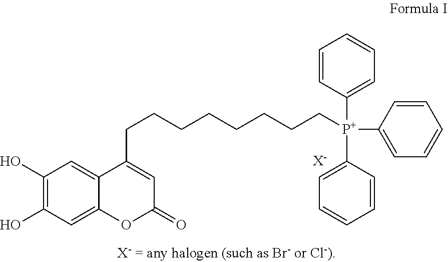

In one preferred embodiment, X⁻ is Br⁻ or Cl⁻. In a more preferred embodiment, X⁻ is Br⁻. In one preferred embodiment, the method comprises topically applying on the affected area of the subject a therapeutically effective concentration of a compound of formula I.

In one embodiment, the compound of Formula I is Compound 1:

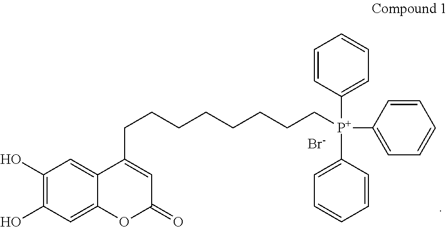 [SMILES: O=c1cc(CCCCCCCC[P+](c2ccccc2)(c2ccccc2)c2ccccc2)c2cc(O)c(O)cc2o1 ; [Br-]]

### BRIEF DESCRIPTION OF THE DRAWINGS

FIG. 1 are photographs showing the effect on day 1, 3, 6, 9, 10 and day 26 after daily topical application from day 1 to day 26 of a cream based vehicle (Vehicle) to a wound on the surface of the skin of diabetic (db/db) mice (Vehicle Control Group)

FIG. 2 are photographs showing the effect on day 1, 3, 6, 9 and day 10 of Compound 1 (0.625% w/w) prepared in the Vehicle topically applied daily from day 1 to day 10 to a wound on the surface of the skin of diabetic (db/db) mice.

FIG. 3 are photographs showing the effect on day 1, 3, 6, 9, 10 and day 26 of Compound 1 (2.5%) prepared in the Vehicle topically applied daily from day 1 to day 26 to a wound on the surface of the skin of diabetic (db/db) mice.

FIG. 4a are photographs showing the effect of topical application of a) Compound 1 (at each concentration of 0.625%, 1.25% and 2.5%) prepared in the Vehicle, b) Compound 2 (2.5%) prepared in the Vehicle and c) Vehicle, to a wound on and around the wound area on the surface of the skin of diabetic (db/db) mice on days 0, 3, 6, 9, 12 and 20. FIG. 4b is a graph showing percentage temporal wound closure (Y-axis) from day 0 to day 20 (X-axis) for the above groups.

FIG. 5 are photographs showing the effect of topical application of a) Compound 1 (at each concentration of 0.625%, 1.25% and 2.5%) prepared in the Vehicle, b) Compound 2 (2.5%) prepared in the Vehicle and c) Vehicle to a wound and the effect on hair growth on and around the wound area on the surface of the skin of non-diabetic (C57) mice on days 0, 10 and 20.

FIG. 6 are photographs showing the effect of topical application of a) Vehicle and b) Compound 1 (2.5%) prepared in the Vehicle to a surgical incision wound (incision wound depth of 2 mm and incision wound length of 3 cm) on the surface of the skin of a rabbit at days 0, 1-8 and 18.

FIG. 7 are photographs showing the effect of topical application of a) Vehicle and b) Compound 1 (2.5%) prepared in the Vehicle, to a wound on the surface of the skin of a rabbit 20 days after treatment.

FIG. 8 are photographs showing the effect of topical administration of a) Vehicle, b) Compound 1 (0.625%) prepared in the Vehicle, c) a combination of Compound 1 (0.625%) and EX-527 (6-chloro-2,3,4,9-tetrahydro-1H-carbazole-1-carboxamide, also known as selisistat, a SIRT1 inhibitor) (0.025%) prepared in a Vehicle and d) EX-527 (0.025%) prepared in Vehicle, on the healing of a surgical incisional wound in a diabetic (db/db) mice model on days 0, 4 and 7.

FIG. 9 are photographs showing the measurement of tensile strength of the skin tissues measured on the 8^{th} day after a treatment for 7 days as described in Example 5 and Table 3.

FIG. 10 depicts psoriatic area and severity index (PASI) scoring for erythema, scaling, skin thickness and ear thickness and percentage loss in the body weight in a psoriasis model.

FIG. 11 depicts phenotypic images of the mouse dorsal skin of representative mice from different treatment groups as described in Example 6 on days 0, 2, 4, 6, and 7.

FIG. 12 depicts the levels of pro-inflammatory cytokines IL-17 and IL 23 (pg/mL) in skin tissue measured using Enzyme Linked Immuno Sorbent Assay (“ELISA”) as described in Example 6.

FIG. 13 depicts hematoxylin and eosin (“H&E”) staining of the dorsal skin of representative mice from different treatment groups (10× and 40× magnification) as described in Example 6.

### DETAILED DESCRIPTION OF THE INVENTION

As utilized in accordance with the present disclosure, unless otherwise indicated, all technical and scientific terms shall be understood to have the same meaning as commonly understood in the art. Unless otherwise required by context, singular terms shall include pluralities and plural terms shall include the singular. The use of any and all examples, or exemplary language provided herein, is intended merely to better illustrate the embodiments and does not pose a limitation on the scope of the claims unless otherwise stated.

The term “wound” refers to any trauma to the tissue of skin of a subject resulting in interruption of continuity within the tissue and wherein the skin is torn, punctured or cut. Wounds typically include, e.g., incisions, scratches, lacerations, chops, cuts, abrasions, puncture wounds, traumatic skin injury, burns, and penetration wounds. A wound may be chronic, e.g. disease or other chronic wounds causing tissue injury such as diabetic wounds and diabetic foot ulcers, pressure sores or pressure ulcers, venous leg ulcers; or acute, e.g., wounds caused by an accident, injury or a surgery caused wound. Medical procedures may also cause wounds such as a dermatological or a cosmetic surgery procedure.

The term “scar” refers to a mark left on the skin tissue where a wound has not healed completely, and a fibrous connective tissue has developed on and around the wound. This includes the formation of such mark or the fibrous connective tissue during or after healing of the wound. Scar symptoms include, e.g., skin scar discoloration (including redness or pigmentation changes), erythema, dryness, peeling, or itchy skin, protruding above the surrounding skin region, keloid formation, month thick bar, scar pain, reduction of scarring and/or surrounding tissue vascularity, reduced flexibility, and poor aesthetic appearance (including scar tissue mass and texture). A scar caused by a wound is also treatable according to the present invention.

The term “wound healing” or “healing of a wound” refers to the restoration of tissue integrity and the terms may be used interchangeably. It will be understood that these terms can refer to a partial or a full restoration of tissue integrity. Treatment of a wound thus refers to the promotion, improvement, progression, expedition, acceleration, or otherwise advancement of one or more stages or processes associated with the wound healing process. Impaired wound healing for the purpose of this invention includes any and all causes, procedures that may impair, inhibit, interfere or delay in the promotion, improvement, progression, expedition, acceleration, or otherwise advancement of one or more stages or processes associated with wound healing. Impaired wound healing as described herein includes that caused by a chemotherapeutic or anti-angiogenic drug.

The terms “therapeutically effective amount” and “therapeutically effective concentration” herein refer to the amount or concentration of active compound or pharmaceutical agent that elicits a biological or medicinal response in a tissue, system, animal, individual or human, which includes one or more of the following: (1) preventing, suppressing, or delaying the disease; for example, preventing, suppressing, or delaying a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

All concentrations of active ingredients, such as Compound 1, in a topical composition are, unless otherwise specified, in percent by weight, based upon 100% total weight of the composition.

The compounds of formula I can be prepared as described in U.S. Pat. No. 9,580,452.

Various embodiments of the present invention are described hereinafter.

In one aspect, the present invention relates to a compound of formula I for use in the treatment of one or more conditions selected from the group consisting of a wound, impaired healing of a wound, hair loss on and around a wound, scars and/or wrinkles on and around a wound and psoriasis by topical application (for example, to the affected area of skin or the wound) to a subject in need thereof.

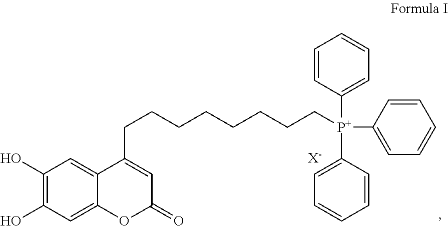

wherein X⁻ is any halogen (such as Br⁻ or Cl⁻). In one preferred embodiment, X⁻ is Br⁻ or Cl⁻. In a more preferred embodiment, X⁻ is Br⁻.

In one embodiment, the present invention relates to the compound of formula I for use in the treatment of a wound.

In another embodiment, the present invention relates to the compound of formula I for use in the treatment of impaired wound healing.

In a further embodiment, the present invention relates to the compound of formula I for use in the treatment of hair loss on and around a wound. The treatment of hair loss on and around a wound may be characterized by promoting and/or expediting the growth of hair on and around a wound site.

In yet another embodiment, the present invention relates to the compound of formula I for use in the treatment of scars and/or wrinkles on and around a wound. The treatment of scars and/or wrinkles may be characterized by minimizing the appearance of scars and/or wrinkles on and around the wound site.

The wound to be treated using a compound of formula I according to any one of the embodiments described herein may be a diabetic wound, an incisional wound, a surgical wound, an accidental wound, a pressure ulcer or bedsore, a diabetic foot ulcer, pyoderma gangrenosum, a burn, a lesion, a cut, or any combination thereof.

In another embodiment, the present invention relates to the compound of formula I for use in the treatment of impaired healing of a wound, wherein the wound healing is impaired due to concomitant administration of one or more medication(s), such as, e.g., immunosuppressants, chemotherapeutics, anti-coagulants, NSAIDs, platelet aggregation inhibitors, anti-angiogenic drugs, and any combination thereof.

In another embodiment, the present invention relates to the compound of Formula I for use in the treatment of psoriasis.

In a preferred embodiment of any of the uses or methods described herein, the compound of formula I is Compound 1:

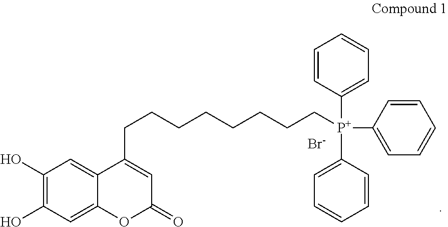 [SMILES: O=c1cc(CCCCCCCC[P+](c2ccccc2)(c2ccccc2)c2ccccc2)c2cc(O)c(O)cc2o1 ; [Br-]]

In another aspect, the present invention relates to a method of treating a wound, impaired healing of a wound, hair loss on and around a wound, scars and/or wrinkles on and around a wound, psoriasis, or any combination thereof, in a subject in need thereof. The method comprises administering (e.g., topically applying on the affected area) a therapeutically effective amount of a compound of formula I (such as a compound of formula 1, where X is Br). In one preferred embodiment, the method comprises topically applying on the affected area of the subject a therapeutically effective concentration of a compound of formula I.

In one embodiment, the present invention relates to a method of treating a wound in a subject in need thereof comprising administering (e.g., topically applying) to the subject a therapeutically effective amount of a compound of formula I (such as Compound 1). In one preferred embodiment, the method comprises topically applying on the affected area of the subject a therapeutically effective concentration of a compound of formula I.

In another embodiment, the present invention relates to a method of treating impaired wound healing in a subject in need thereof comprising administering (e.g., topically applying) to the subject a compound of formula I. In one preferred embodiment, the method comprises topically applying on the affected area of the subject a therapeutically effective concentration of a compound of formula I.

In a further embodiment, the present invention relates to a method of treating hair loss on and around a wound in a subject in need thereof comprising administering (e.g., topically applying) to the subject a compound of formula I. The treatment of hair loss on and around a wound may be characterized by promoting and/or expediting the growth of hair on and around a wound site. In one preferred embodiment, the method comprises topically applying on the affected area of the subject a therapeutically effective concentration of a compound of formula I.

In yet another embodiment, the present invention relates to a method of treating a scar(s) and/or wrinkle(s) on and around a wound in a subject in need thereof comprising administering (e.g., topically applying) to the subject a compound of formula I. The treatment of scars and/or wrinkles may be characterized by minimizing the appearance of scars and/or wrinkles on and around a wound site. In one preferred embodiment, the method comprises topically applying on the affected area of the subject a therapeutically effective concentration of a compound of formula I.

In any of the methods of treating a wound, impaired healing of a wound, hair loss on and around a wound or scars and/or wrinkles on and around a wound described herein, the wound may be a diabetic wound, an incisional wound, a surgical wound, an accidental wound, a pressure ulcer/bedsore a diabetic foot ulcer, pyoderma gangrenosum, a burn, a lesion or a cut.

In another embodiment, the present invention relates to a method of treating psoriasis comprising administering (e.g., topically applying) to a subject in need thereof a compound of formula I. In one preferred embodiment, the method comprises topically applying on the affected area of the subject a therapeutically effective concentration of a compound of formula I (such as Compound 1).

In a preferred embodiment of any of the methods described herein, the compound of formula I is Compound 1:

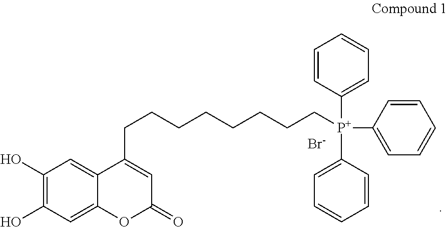 [SMILES: O=c1cc(CCCCCCCC[P+](c2ccccc2)(c2ccccc2)c2ccccc2)c2cc(O)c(O)cc2o1 ; [Br-]]

In any of the embodiments described herein, the wound is present on the surface of the skin of a subject. In one embodiment the subject is a mammal. In a preferred embodiment, the subject is a human. The methods described herein are useful in both human therapeutics and veterinary applications. For veterinary purposes, the term “subject” includes, but is not limited to, farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

Another embodiment relates to a topical composition and kits comprising a therapeutically effective concentration or amount of a compound of formula I and optionally a pharmaceutically acceptable excipient. The compositions described herein are useful for the treatment of a wound, impaired healing of a wound, hair loss on and around a wound, scars and/or wrinkles on and around a wound, psoriasis, or any combination thereof.

In one embodiment, the present invention relates to a topical composition comprising a therapeutically effective amount of a compound of formula I for the treatment of a wound and optionally a pharmaceutically acceptable excipient.

In another embodiment, the present invention relates to a topical composition comprising a therapeutically effective amount of a compound of formula I and optionally a pharmaceutically acceptable excipient for the treatment of impaired wound healing.

In one embodiment, the present invention relates to a topical composition comprising a therapeutically effective amount of a compound of formula I and optionally a pharmaceutically acceptable excipient for the treatment of hair loss on and around a wound. The treatment of hair loss on and around a wound may be characterized by promoting and/or expediting the growth of hair on and around a wound site.

In yet another embodiment, the present invention relates to a topical composition comprising a therapeutically effective amount of a compound of formula I and optionally a pharmaceutically acceptable excipient for the treatment of scars and/or wrinkles on and around a wound. The treatment of scars and/or wrinkles may be characterized by minimizing the appearance of scars and/or wrinkles on and around a wound site.

In another embodiment, the present invention relates to a topical composition comprising a therapeutically effective amount of a compound of formula I and optionally a pharmaceutically acceptable excipient for the treatment of psoriasis.

In a preferred embodiment of any of the compositions described herein, the compound of formula I is Compound 1.

Any of the topical compositions described herein may further comprise one or more suitable pharmaceutically acceptable excipients, known to those of ordinary skill in the art. The composition may be in the form of a topical dosage form, e.g., a solution, gel, ointment, cream, lotion, paste, spray foam or aerosol. The compound of formula I (such as Compound 1) may be made into topical compositions with appropriate pharmaceutically acceptable carriers or diluents and may be formulated into semi-solid or liquid forms.

According to another aspect, the present invention relates to a kit comprising a therapeutically effective amount of a compound of formula I (e.g., in the form of a topical composition) and, optionally, instructions for using the kit. The kits described herein are useful for the treatment of a wound, impaired healing of a wound, hair loss on and around the wound, scars and/or wrinkles on and around the wound, psoriasis, or any combination thereof.

The following examples are illustrative of the specific embodiments of the disclosure described above. They are set forth for explanatory purposes only and should not be construed as limiting the scope of the disclosure in any way.

### EXAMPLES

General Methods and Procedures

Preparation of Test Compounds:

For the purpose of animal studies in the examples below, the topical preparation of the test compounds was made in a vehicle comprising an emulsifying ointment, a preservative (p-chloro cresol) and water. The vehicle was devoid of any test compound. The test compounds (Compounds 1 and 2) were incorporated into the aqueous cream base preparation by trituration. The aqueous cream was made using emulsifying ointment BP, p-chlorocresol (preservative) and water as per the procedure of British Pharmacopoeia. Control formulations are devoid of any active ingredient.

Step 1: Preparation of Emulsifying Ointment

An emulsifying ointment was prepared by using emulsifying wax, white soft paraffin and liquid paraffin. These ingredients were melted and warmed up to 60° C. in a water bath, then stirred continuously until it became cool.

Step 2: Preparation of Anionic Emulsifying Cream

The emulsifying ointment was melted and warmed up to 60° C. in water bath and the required quantity of para-chlorocresol was dissolved in cold water (chlorocresol is soluble in cold water), the temperature was checked using a thermometer and purified water was added to the melted ointment and stirred continuously until it became cool.

Step 3: Preparation of Compound 1 and 2 Cream Formulation

The required quantity of Compound 1 or 2 was added to the cream and mixed with a homogenizer. After proper mixing of the medicament, the formulation was transferred into a suitable container and preserved at 4° C.

### Emulsifying Ointment Base Preparation

| S. No. | Ingredients | Qty (100 gms) | Qty (20 gms) |
|---|---|---|---|
| 1 | Emulsifying wax | 30 | 6 |
| 2 | White soft paraffin | 50 | 10 |
| 3 | Liquid paraffin | 20 | 4 |

### Control Formulation

| S. No. | Ingredients | Qty (20 gms) |
|---|---|---|
| 1 | Emulsifying ointment | 6 gms |
| | base preparation | |
| 2 | p-chloro cresol | 0.02% |
| 3 | Purified water | 13.98 gms |

### Formulation 1—2.5% Compound 1 Cream

| S. No. | Ingredients | Qty (20 gms) |
|---|---|---|
| 1 | Compound 1 | 500 mg (2.5%) |
| 2 | Emulsifying ointment | 6 gms |
| | base preparation | |
| 3 | p-chloro cresol | 0.02% |
| 4 | Purified water | 13.48 gms |

### Formulation 2—2.5% Compound 2 Cream

| S. No. | Ingredients | Qty (20 gms) |
|---|---|---|
| 1 | Compound 2 | 500 mg (2.5%) |
| 2 | Emulsifying ointment | 6 gms |
| | base preparation | |
| 3 | p-chloro cresol | 0.02% |
| 4 | Purified water | 13.48 gms |

### Formulation 3—1.25% Compound 2 Cream

| S. No. | Ingredients | Qty (20 gms) |
|---|---|---|
| 1 | Compound 2 | 250 mg (1.25%) |
| 2 | Emulsifying ointment | 6 gms |
| | base preparation | |
| 3 | p-chloro cresol | 0.02% |
| 4 | Purified water | 13.73 gms |

### Formulation 4—0.625% Compound 2 Cream

| S. No. | Ingredients | Qty (20 gms) |
|---|---|---|
| 1 | Compound 2 | 125 mg (0.625%) |
| 2 | Emulsifying ointment | 6 gms |
| | base preparation | |
| 3 | p-chloro cresol | 0.02% |
| 4 | Purified water | 13.85 gms |

### Example 1: Effect of Compound 1 on Diabetic Wound Healing Process and Hair Growth on and/or Around the Wound Area in Diabetic (db/db) Mice Model

Method:

In this study, mice of db/db strain (age: 13-14 weeks) were used as this strain is more analogous to diabetic wound healing in humans. A 1 cm² wound was made using a punch biopsy on the dorsal side of the db/db mice under local anesthetic conditions. The animals were divided into 5 groups with 5 animals in each group as follows:
Group 1 (n=5): treated with vehicle alone (Diabetic Control Group).
Group 2 (n=5) treated with Compound 2 (2.5%) prepared in the vehicle.
Group 3 (n=5): treated with Compound 1 (0.625%) prepared in the vehicle.
Group 4 (n=5) treated with Compound 1 (1.25%) prepared in the vehicle.
Group 5 (n=5) treated with Compound 1 (2.5%) prepared in the vehicle.

Three concentrations of Compound 1, i.e. 0.625%, 1.25%, and 2.5%, were tested for this study. The topical preparations were applied on the wound once daily for 12 days. Images of the wound area were visualized, and measurements of the wound area were recorded on each day of the study.

Results:

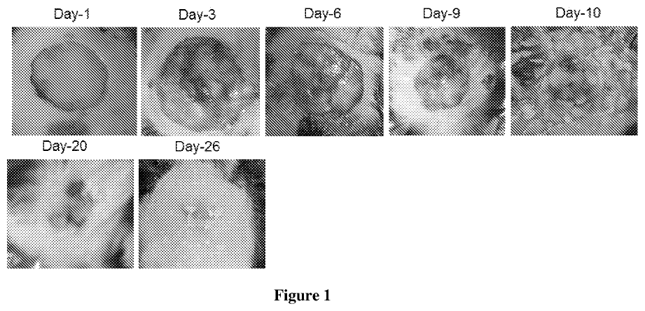

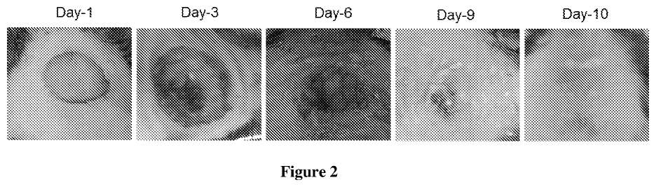

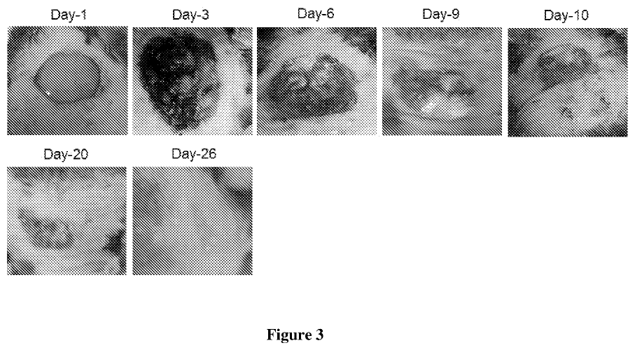

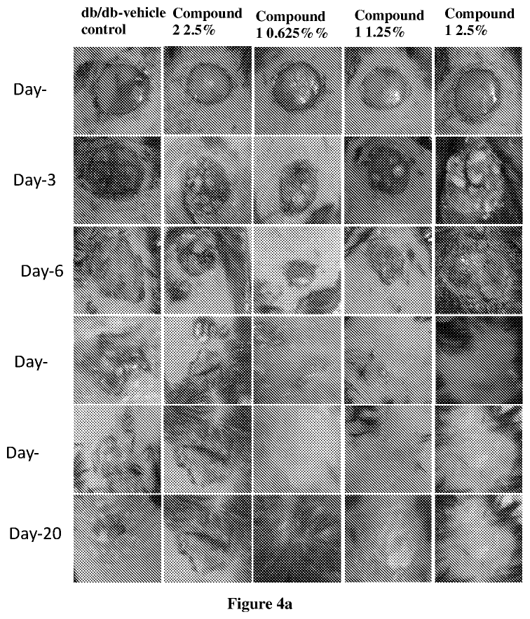

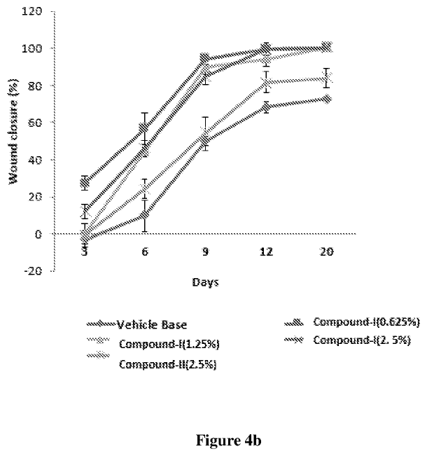

The Diabetic Control Group and Group 2 showed wound closure by day 26 (See FIGS. 1 and 3). Complete closure of the wound was observed by day 10 in Groups 3-5 (See FIGS. 2 and 4a and 4b (percentage temporal wound closure)). Table 1 shows the percentage wound healing observed in each group.

TABLE 1
db-db mice wound healing observations

| | | Wound healing | |
|---|---|---|---|
| | Number | observed | |
| | of animals | animals in a | % of Wound |
| Group | in the group | group on day 10 | healing |
| Group 1 (Diabetic | 5 | 0 | 0 |
| Control Group) | | | |
| Group 2 (2.5%) | 5 | 0 | 0 |
| Group 3 (0.625%) | 5 | 5 | 100 |
| Group 4 (1.25%) | 5 | 4 | 90 |
| Group 5 (2.5%) | 5 | 4 | 90 |

### Example 2: Effect of Compound 1 on Wound Healing and Hair Growth on and/or Around the Wound Area in Non-Diabetic C-57 Mice Model

Method:

In this study, C57BL/6j mice strain (age: 12 weeks) were used for studying the wound healing process. A 1 cm² wound was made using a punch biopsy on the dorsal side of the C57 mice. The animals were divided into 5 groups consisting of 5 animals in each group as follows:
Group 6 (n=5): treated with vehicle alone (Non-Diabetic Control Group).
Group 7 (n=5) treated with Compound 2 (2.5%) prepared in the vehicle.
Group 8 (n=5): treated with Compound 1 (0.625%) prepared in the vehicle.
Group 9 (n=5) treated with Compound 1 (1.25%) prepared in the vehicle.
Group 10 (n=5) treated with Compound 1 (2.5%) prepared in the vehicle.

Three concentrations of Compound 1, i.e. 0.625%, 1.25%, and 2.5%, prepared in the vehicle were tested in this study. The topical preparations were applied on the wound once daily for 12 days starting 12 hours after the wound was created. Images of the wound area were visualized, and measurements of area were recorded on each day of the study.

Result:

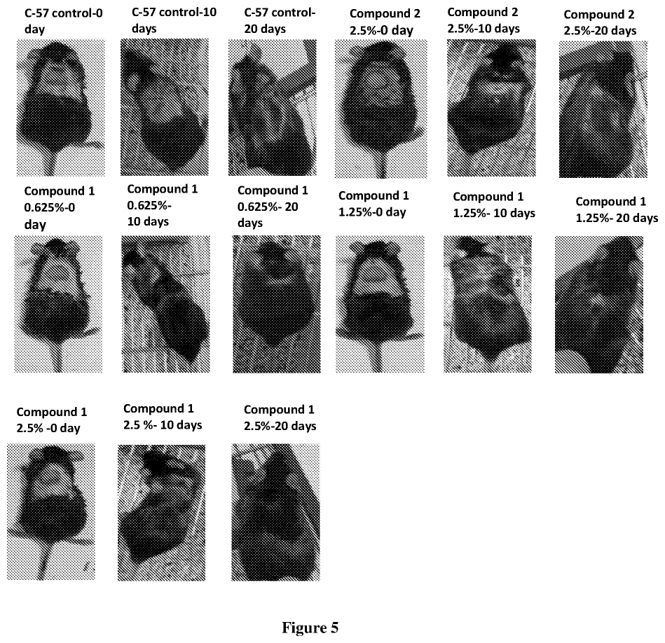

The Non-Diabetic Control Group and Group 6 showed wound closure by day 14 (See FIG. 5). Complete closure of the wound was observed in Groups 8-10 from day 9 to day 12. Table 2 shows the percentage wound healing observed in each group.

TABLE 2
C57BL/6j mice wound healing observations

| | Number | Wound healing | % of |
|---|---|---|---|
| | of animals | observed animals in | Wound |
| Group name | in the group | a group | healing |
| Group 6 | 5 | 4 | 90 |
| (Non-Diabetic | | | |
| Control Group) | | | |
| Group 7 | 5 | 4 | 90 |
| Group 8 | 5 | 4 | 90 |
| Group 9 | 5 | 4 | 90 |
| Group 10 | 5 | 5 | 100 |

### Example 3: Effect of Compound 1 on Incisional Wound Healing in Rabbit

Method:

In this study, New Zealand rabbits weighing between 2.0-2.5 kg body weight were used and divided into two groups;

Control Group: Treated with the vehicle alone.

Test Group: Treated with Compound 1 (2.5%) prepared in the vehicle.

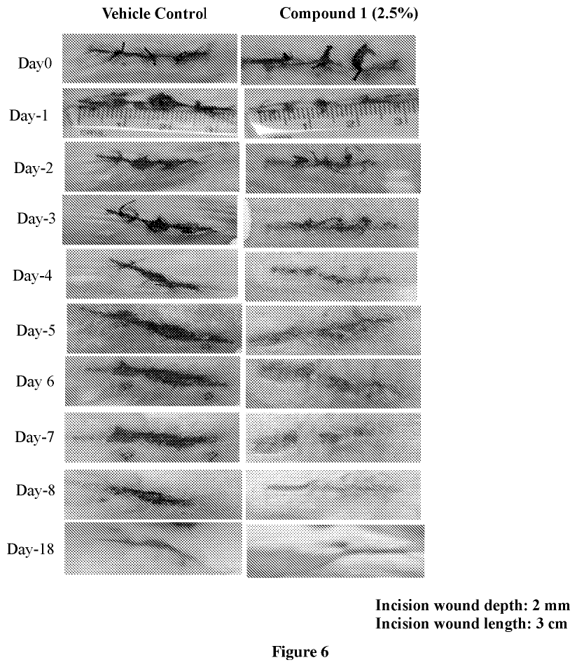

Para vertebral straight incisions of 2 cm length each were made through the entire thickness of the skin on either side of the vertebral column with the help of a sharp scalpel. After complete haemostasis, the wound was closed by means of interrupted sutures placed at equidistance points about 1 cm apart (See FIG. 6). Animals were treated once daily from day 0 to day 14 post-wounding day. Images were taken daily during the study period. Sutures were removed on day 7 and observed for wound closure.

Results:

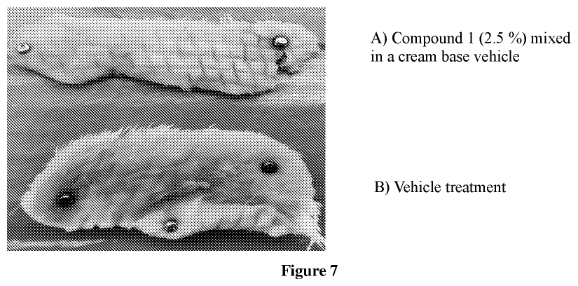

The Test Group rabbits were compared with the vehicle base Control Group rabbits for wound closure at each time point during the study. Faster surgical wound healing was observed within 8 days in the Test Group rabbits when compared to the untreated Control Group rabbits. No surgical scar was observed in the Test Group rabbits as compared to the Control Group rabbits. See FIG. 7.

### Example 4: Effect of Compound 1 on Incisional Wound Healing in Diabetic Mice (db/db) Model

Method:

In this study, mice of db/db strain (age: 12 weeks) were used. The animals were divided into four groups:
Group 11: Mice control treated with the vehicle alone.
Group 12 Mice treated with Compound 1 (0.625%) prepared in the vehicle.
Group 13 Mice treated with Compound 1 (0.625%) and EX-527 (0.025%) in the
vehicle.
Group 14: Mice treated with EX-527 (0.025%) in the vehicle.

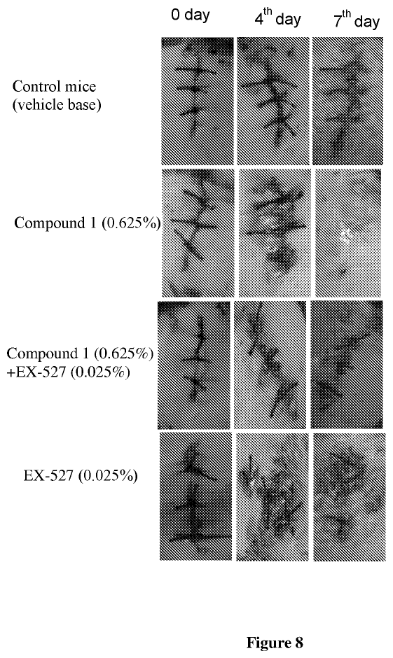

Para vertebral straight incisions of 2 cm length and 1 mm depth each were made through the entire thickness of the skin, on either side of the vertebral column with the help of a sharp scalpel. After complete haemostasis, the wound was closed by means of interrupted sutures placed at equidistance points about 1 cm apart (See FIG. 8) All the groups were treated once daily for 7 days with the respective test and control preparations. Images of the wound area were taken on each day of the study by photographs and Doppler imaging. The wound area was measured using a transparent tracing sheet before and during the treatment to measure the wound closure at every 4 day interval. Sutures were removed on day 7 and the animals were observed for wound closure.

Results:

The Group 12 mice showed complete healing of the incisional wound within 7 days as compared to the Group 11, 13 and 14. mice. See FIG. 8. Group 13, which was treated with EX-527 (0.025%) an SIRT1 inhibitor, in combination with Compound 1, hindered the wound healing process induced by Compound 1. This study suggests that Compound 1 induces wound healing by enhancing SIRT1 activity. This result supports the hypothesis that Compound 1 promotes in vitro angiogenesis tube formation, which is perturbed in the presence of EX-527.

### Example 5: Measurement of Tensile Strength of the Skin Tissues on Day 8, after Treatment for 7 Days

Method:

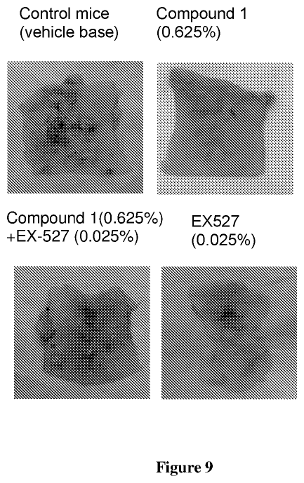

Fresh mice skin was treated once daily and stretched between the two ends of the tensile machine with a load of 100 kg with a stretching speed of 5 mm/min. (FIG. 9). The results are shown in Table 3.

TABLE 3
Tensile Strength Observations

| Group-ID | Tensile strength (N) |
|---|---|
| Control Mice | 5.2 ± 1.08 |
| Compound 1 (0.625%) | 55.33 ± 1.7 |
| Compound 1(0.625%) + | 5.7 ± 1.62 |
| EX527 (0.025%) | |
| EX527 (0.025%) | 6.03 ± 2.51 |

### Example 6: Effect of Topical Application of Compound 1 in Improving IMQ-Induced Psoriasis in Balb/c Mice

Method:

The effect of topical administration of Compound 1 for the treatment of psoriasis was tested in this animal study. In this study, typical features of psoriasis, namely erythema, scaling, and skin thickness, were induced by the application of 5% w/w imiquimod (IMQ) cream on the dorsal skin of Balb/c mice over a period of 6 consecutive days. Animals that developed psoriasis-like symptoms were used for studying the efficacy of the Compound 1 prepared in a vehicle (as described above). In this study, male Balb/c mice (aged 6 to 8 weeks) were used. The animals were divided into 8 groups consisting of 10 animals in each group as follows:

Sham Control Group:

Sham Control: The back of the mice were shaved and no treatment was given to the animals. This group served as a control.

IMQ Control Group:

Animals in this group were topically treated with 62.5 mg of commercially available imiquimod cream (5%) on the shaved back for 6 consecutive days (day 1 to 6 of study) at a specific time in the morning. This negative control group represents the typical psoriatic model induced by imiquimod 5% (IMQ).

IMQ+Vehicle Base Group:

This group represents the mice induced with psoriasis by imiquimod followed, after 12 hours, by treatment with the vehicle used for the preparation of compound of Formula I on the shaved back of the animals from day 1 to day 6 of the study.

IMQ+Clobetasol Propionate Cream 0.05%:

This group represents the mice induced with psoriasis by imiquimod followed, after 12 hours, by treatment the commercially available clobetasol propionate (0.05%) cream (20 μg/2 cm² area) on the shaved back of the animals from day 1 to day 6 of the study.

IMQ+Compound 1 (0.313%) Group:

This group represents the mice induced with psoriasis by imiquimod followed, after 12 hours, by treatment with Compound 1 (0.313%, which corresponds to 82.6 mg/cm² area) prepared in the vehicle on the shaved back of the animals from day 1 to day 6 of the study.

IMQ+Compound 1 (0.625%) Group:

This group represents the mice induced with psoriasis by imiquimod followed, after 12 hours, by treatment with Compound 1 (0.625%, which corresponds to 82.6 mg/cm² area) prepared in the vehicle on the shaved back of the animals from day 1 to day 6 of the study.

The activity schedule of this study is described in Table 4 below:

TABLE 4
Activity Schedule

| | Day | |
|---|---|---|
| Activity | 1 to 6 | Day 7 |
| Body weight | √ | √ |
| Treatment (Topical) (12 hr after IMQ) | √ | |
| Imiquimod application | √ | |
| Erythema and scaling scoring | √ | √ |
| Skin collection, spleen, liver weight | | √ |
| Cytokines (IL-17 and IL-23) estimation | | √ |
| Histopathology of skin | | √ |

Histopathology Study:

As a part of the efficacy study, a histopathology study was performed to study the morphological observations of the skin of mice representative of the different treatment groups. At the end of experiment, skin samples of mice from each group were fixed in 4% paraformaldehyde and embedded in paraffin. From each paraffin block, 4 μm thick sections were made and mounted on the glass slides. The sections were stained by haematoxylin and eosin and images were taken and evaluated.

Results:

Psoriatic area and severity index (PASI) scoring was analyzed to determine the efficacy of compositions containing a Compound 1 with appropriate controls as part of the study. The severity of inflammation of the skin was evaluated by an objective scoring system based on the clinical PASI scores. Erythema (0-4) and scaling (0-4) were scored independently where, 0-none; 1-slight; 2-moderate; 3-marked; 4-very marked. The cumulative score (erythema plus scaling) served as a measure of the severity of inflammation (scale 0-8).

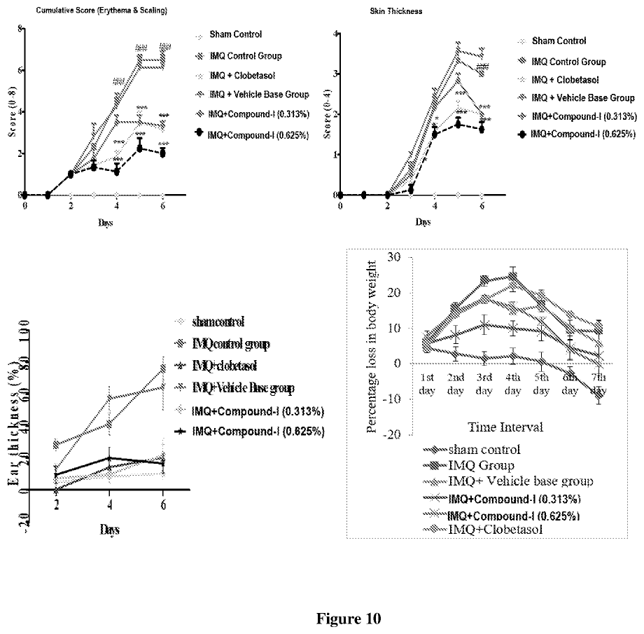

FIG. 10 depicts PASI scoring including body weight, erythema, scaling, and skin thickness determined during the study. Individual body weights of all mice were recorded daily. The loss in body weight was calculated with respect to the body weight at day 0. The negative control group, which represents the typical psoriasis condition, showed the highest PASI score with respect to all the parameters. The treatment groups, which include groups treated with Compound 1 (0.313% and 0.625%) prepared in vehicle showed declines in PASI scores over the treatment period, almost similar to the positive control. The IMQ+Compound 1 (0.625%) group showed better declines in PASI scores when compared to the IMQ+Compound 1 (0.313%) group and the positive control group. Based on PASI scoring, the anti-psoriatic effect visible from the treatment groups was comparable to the positive control group which was treated with the commercially available clobetasol propionate cream.

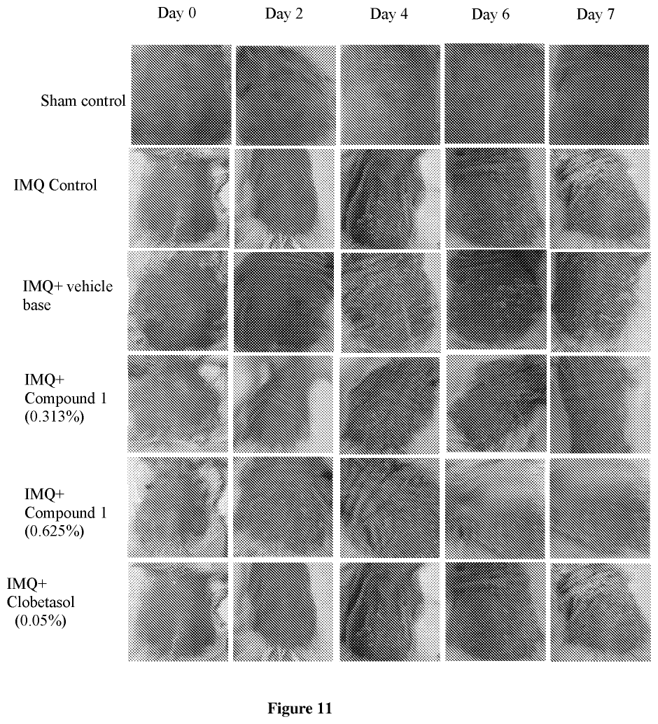

FIG. 11 depicts the images of mice representing different groups of the study, namely sham, negative control, positive control, and treatment groups treated with preparations of Compound 1 of different concentrations from day 0 to day 7.

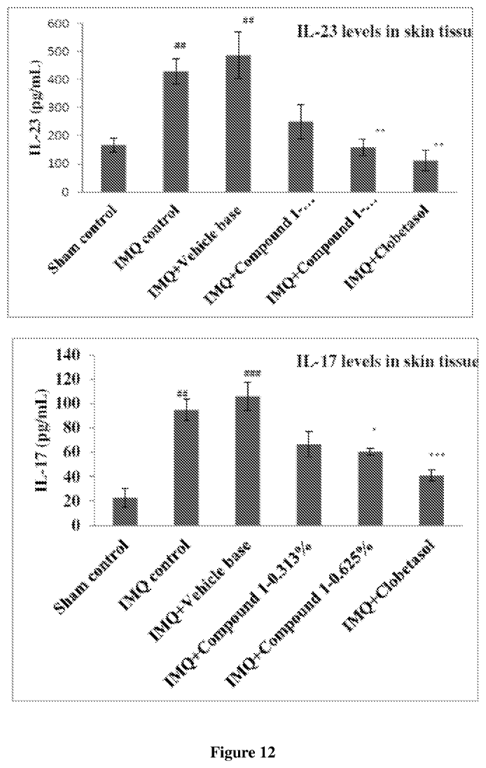

An IMQ-induced psoriasis model reproduces biochemical and histopathological parameters characteristic of human psoriatic lesions. Topical application of the IMQ cream increased levels of cytokines including IL-23 and IL-17 in the treated skin tissues. The animals were euthanized on day 7. Spleen, liver and skin tissues were collected from all the animals Spleen and liver tissues were weighed. IL-17 and IL-23 levels were measured by ELISA using a commercially available kit. The skin homogenates obtained from mice of different groups were analyzed using ELISA for quantification of IL-17 and IL-23 levels as the selected interleukins are the typical inflammatory markers of psoriasis. As illustrated in FIG. 12, the IMQ group (p<0.01) and IMQ+vehicle base (p<0.001) group showed a significant increase in IL-17 levels when compared to sham group. A decline in IL-17 levels was seen in all treatment groups when compared to the negative control. The IMQ group and IMQ+vehicle base group showed a significant increase in IL-23 levels (p<0.01) when compared to the sham control group. A decline in IL-23 levels was seen in the treatment groups, including IMQ+Compound 1 (0.313% and 0.625%) and IMQ+clobetasol (p<0.01) groups when compared to the negative control. Compared to the sham control group, the IMQ group exhibited significant elevation of interleukin levels (p<0.01) which reflected the development of psoriatic inflammation.

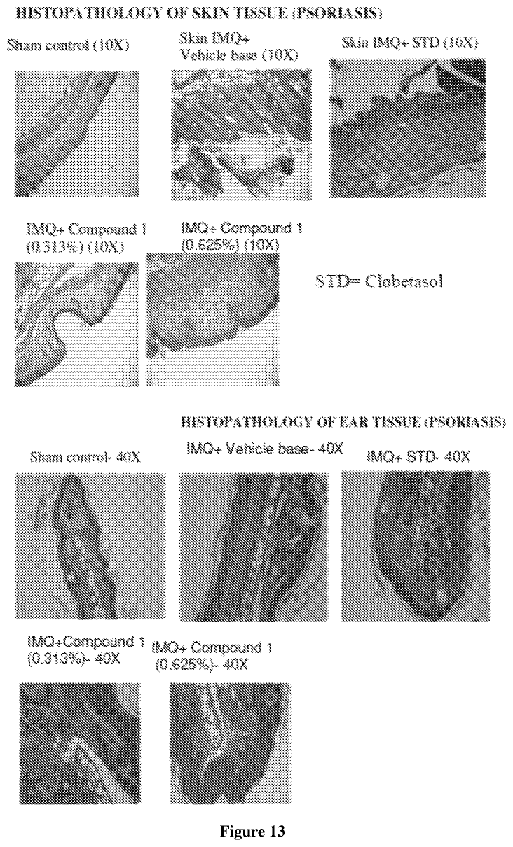

The negative control group of the study (imiquimod-treated animals) represents the typical psoriatic features acanthosis, parakeratosis, hyperkeratosis, and dermal infiltrate upon H&E staining. Similar staining of skin collected from mice of the treatment groups showed a decline in the thickening of epidermis, decline in thickening of the stratum corneum, and a lessened number of dermal infiltrates. The histopathological images are depicted in FIG. 13. Histopathological results from both skin and ear shows that psoriasis induced by IMQ was ameliorated more by Compound 1 preparation (0.625%) and was comparable to the effect of the clobetasol propionate cream.

1. A method of treating a wound, impaired healing of a wound, hair loss on and around a wound, scars and/or wrinkles on and around a wound, psoriasis, or any combination thereof, in a subject in need thereof, the method comprising topically administering to an affected area of the subject a therapeutically effective amount of a compound of formula I: 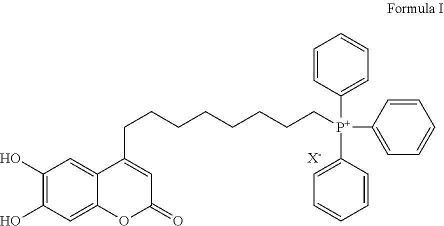
wherein X⁻ is any halogen.

2. The method of claim 1, wherein the wound is selected from a diabetic wound, an incisional wound, a surgical wound, an accidental wound, a pressure ulcer/bedsore, a diabetic foot ulcer, pyoderma gangrenosum, a burn, a lesion or a cut.

3. The method of claim 1, wherein the method treats a wound.

4. The method of claim 1, wherein the method treats impaired healing of a wound.

5. The method of claim 1, wherein the method treats hair loss on and around a wound.

6. The method of claim 1, wherein the method treats scars and/or wrinkles on and around a wound.

7. The method of claim 1, wherein the method treats psoriasis.

8. The method of claim 6, wherein the treatment is characterized by reducing, minimizing or disappearing the appearance of scars and/or wrinkles on and around the wound site.

9. The method of claim 5, wherein the treatment is characterized by promoting and/or expediting the growth of hair on and around a wound site.

10. The method of claim 1, wherein X⁻ is Br⁻ or Cl⁻.

11. The method of claim 1, wherein the compound of formula I is

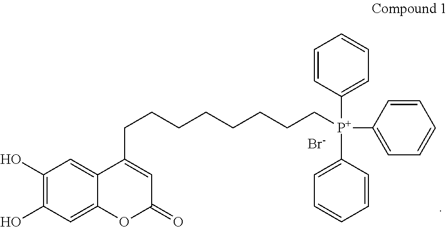 [SMILES: O=c1cc(CCCCCCCC[P+](c2ccccc2)(c2ccccc2)c2ccccc2)c2cc(O)c(O)cc2o1 ; [Br-]]

12-22. (canceled)

23. A topical composition for use in treating a wound, impaired healing of a wound, hair loss on and around a wound, scars and/or wrinkles on and around a wound, psoriasis, or any combination thereof and a pharmaceutically acceptable excipient, the composition comprising a compound of formula I: 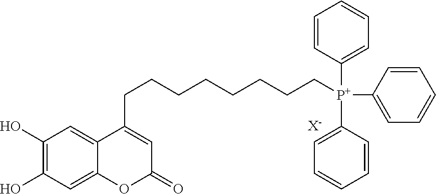
wherein X⁻ is any halogen and a pharmaceutically acceptable excipient.

24. The topical composition of claim 23, wherein X⁻ is Br⁻ or Cl⁻.

25. The topical composition of claim 23, wherein X⁻ is Br—.

26. The topical composition of claim 23, wherein the pharmaceutically acceptable excipient is one or more of an emulsifying agent, preservative, or any combination thereof.

27. A kit for treating a wound, impaired healing of a wound, hair loss on and around a wound, scars and/or wrinkles on and around a wound, psoriasis, or any combination thereof, comprising a compound of formula I: 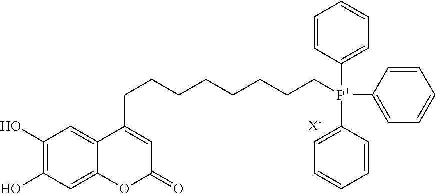
wherein X⁻ is any halogen; and
optionally, instructions for using the kit.

28. The kit of claim 27, wherein X⁻ is Br⁻ or Cl⁻.

29. The kit of claim 27, where X⁻ is Br⁻.
